(19) United States
(12) Patent Application Publication
(10) Pub. No.: US 2022/0288256 A1
(43) Pub. Date: Sep. 15, 2022

(54) STERILIZATION WASHING METHOD AND STERILIZATION WASHING DEVICE

(71) Applicant: JUSBLANCH, CO, LTD, Kanuma-shi, Tochigi (JP)

(72) Inventor: Masayoshi EDA, Kanuma-shi (JP)

(73) Assignee: JUSBLANCH, CO, LTD, Kanuma-shi, Tochigi (JP)

(21) Appl. No.: 16/649,375

(22) PCT Filed: Sep. 20, 2017
(86) PCT No.: PCT/JP2017/033896

§ 371 Date: May 27, 2020

(51) Int. Cl. A61L 2/18; A61L 9/14; B08B 1/04

(52) U.S. Cl. CPC A61L 2/18; A61L 9/14; B08B 1/04

(57) ABSTRACT

The present invention provides a method of disinfection and cleaning capable of, in conjunction with removing, by washing, dust and grit, dirt, and the like adhered to an underside of an object to be disinfected and cleaned such as traveling bags or suitcases, space disinfection as well as a disinfection and cleaning apparatus. The method of disinfection and cleaning includes a step of removing dust and grit and bacteria and the like adhered to the underside of the object to be disinfected and cleaned by contact with a disinfecting and cleaning fluid by means of cleaning and removal means. A disinfection and cleaning apparatus 1 is characterized in that the apparatus includes a disinfection and cleaning apparatus main unit (10); a cleaning and removal means provided in the disinfection and cleaning apparatus main unit; a space on which the object to be disinfected and cleaned can be placed; and a sealing means causing the disinfection and cleaning apparatus main unit to be in a hermetically sealed state, wherein the cleaning and removal means includes a pool unit (102) for storing the disinfecting and cleaning fluid, formed in the bottom of the disinfection and cleaning apparatus main unit; a cleaning mechanism (103) placed in the pool unit; and a drive source (104) for driving the cleaning mechanism in such a way that the cleaning mechanism causes the disinfecting and cleaning fluid to come into contact with the object to be disinfected and cleaned.

### TECHNICAL FIELD

The present invention relates to a method of disinfection and cleaning of an object to be disinfected and cleaned including bags or the like such as traveling bags or suitcases and a disinfection and cleaning apparatus used in the method of disinfection and cleaning; more specifically, the present invention relates to a disinfection and cleaning method for, in conjunction with removing, by washing, dust and grit, dirt, and the like adhered to an underside of the object to be disinfected and cleaned including bags or the like such as traveling bags or suitcases of foreign travelers or the like, removing bacteria and viruses adhered all over the object to be disinfected and cleaned as well as the disinfection and cleaning apparatus.

### BACKGROUND ART

In recent years, means of transportation and communication have been significantly developed and political, economic, and social structures have been internationalized, which has boosted international exchanges. In addition, our country has been putting emphasis on tourism policies and has sought to boost exchanges with foreign citizens and increase the number of international tourists from foreign countries in order to broadly introduce the history and the culture of Japan to the world.

Under such circumstances, bags or the like such as traveling bags or suitcases are used in trips regardless of domestic or overseas, but during use of such a bag or the like, in particular a bag or the like with casters on an underside, dust and grit, dirt and the like often adheres to the casters on the underside. Specifically, traveling bags are often carried into rooms as it is with dust and grit, dirt, and the like adhered to the underside casters thereof, which has been pointed out as hygiene issues. In addition, bacteria and viruses often adhere all over the traveling bag.

Concerning disinfection of bacteria and viruses in such a situation, space disinfection methods have been proposed. For example, Japanese Unexamined Patent Publication (Kokai) No. 2004-275582 (patent literature 1) discloses a disinfecting device that can disinfect an object to be disinfected by equalizing concentration of disinfecting gas in the upper, middle, and lower parts of a storage space and a disinfection room that utilizes the disinfecting device.

In addition, the Gazette of Japanese Utility Model Registration No. 3091374 (patent literature 2) discloses a method in which a chlorine dioxide solution or a gel-like solid matter in which chlorine dioxide is dissolved is disposed in a locker and chlorine dioxide is vaporized to disinfect the interior of the locker.

In addition, Japanese Unexamined Patent Publication (Kokai) No. 2015-93811 (patent literature 3) discloses a simplified space disinfection method using chlorine dioxide and a simplified space disinfection device while Japanese Unexamined Patent Publication (Kokai) No. 2011-152234 (patent literature 4) discloses a chlorine dioxide gas generator equipped with a container having an opening and a storing chlorine dioxide gas generating agent and an aromatic substance for reducing an unpleasant odor of chlorine dioxide.

Japanese Unexamined Patent Publication (Kokai) No. 2010-104860 (patent literature 5) discloses a method in which an object A to be cleaned is immersed as in a dip-washing, a tank is hermetically sealed, an ozone gas is generated in a form of microbubbles to be injected into and mixed with water in the tank to generate ozonated water, and the object A to be cleaned is cleaned by immersion in the ozonated water in which the concentration of dissolved ozone is optimized.

However, in general, there has been little recognition of issues caused by dust and grit and bacteria and the like adhered to casters attached to an underside of a traveling bag with casters and concern was rarely shown for removing the issue. Consequently, there has been no disclosure of a disinfection and cleaning method for removing dust and grit and bacteria and the like adhered to the casters with a cleaning and disinfecting solution and a disinfection and cleaning apparatus. There has been no disclosure of a combination of such a method and an apparatus with the space disinfection method and a space disinfection device, either, and there have been desired a more efficient disinfection and cleaning method and a disinfection and cleaning apparatus.

### PRIOR ART DOCUMENT

### Patent Literature

[PTL 1] Japanese Unexamined Patent Publication (Kokai) No. 2004-275582

[PTL 2] Japanese Utility Model Registration No. 3091374

[PTL 3] Japanese Unexamined Patent Publication (Kokai) No. 2015-93811

[PTL 4] Japanese Unexamined Patent Publication (Kokai) No. 2011-152234

[PTL 5] Japanese Unexamined Patent Publication (Kokai) No. 2010-104860

### SUMMARY OF INVENTION

### Technical Problem

Thus, the first challenge to the present invention is to provide a method of disinfection and cleaning for removing dust and grit, dirt, and bacteria and the like adhered to an underside of an object to be disinfected and cleaned including bags or the like such as traveling bags or suitcases by washing with a disinfecting and cleaning fluid without damaging the object as well as a disinfection and cleaning apparatus used in the method of disinfection and cleaning.

The second challenge to the present invention is to provide a method of disinfection and cleaning additionally including, in conjunction with removing dust and grit, dirt, and bacteria and the like adhered to an underside of an object to be disinfected and cleaned by washing with a disinfecting and cleaning fluid without damaging the object, a method for removing bacteria and viruses adhered all over the object to be disinfected and cleaned as well as a disinfection and cleaning apparatus used in the method of disinfection and cleaning.

### Solution to Problem

Therefore, the inventor of the present invention has intensively studied to solve the challenges to the present invention; as a result, with respect to the first challenge, the inventor found that the challenge can be solved by providing a method of disinfection and cleaning, with respect to dust and grit, dirt, and the like adhered to the underside of the object to be disinfected and cleaned, the method including supplying a disinfecting and cleaning fluid to an underside of an object to be disinfected and cleaned by means of a cleaning and removal means that can come into contact with the underside of the object to be disinfected and cleaned to remove the dust and grit, dirt, and the like adhered to the underside of the object to be disinfected and cleaned and a disinfection and cleaning apparatus including the cleaning and removing means used in the method of disinfection and cleaning; the inventor has focused on this point to conceive the present invention based on the finding.

With respect to the second challenge, the inventor found that the challenge can be solved by performing disinfection and cleaning and space disinfection systematically, i.e., by performing, in conjunction with the method of disinfection and cleaning, with respect to dust and grit, dirt, and bacteria and the like adhered to the underside of the object to be disinfected, the method including bringing the disinfecting and cleaning fluid into contact with dust and grit, dirt, and bacteria and the like adhered to of the underside of the object to be disinfected and cleaned by means of the cleaning and removal means, a method of disinfection using a space disinfection means for removing bacteria and viruses adhered all over the object to be disinfected and cleaned with attaching the space disinfection means inside a disinfection and cleaning apparatus used in the disinfection method, for example, the disinfection and cleaning apparatus described above; the inventor has focused on this point to conceive the present invention based on the finding.

In other words, the present invention provides “hospitality by means of disinfection”, in particular, to users of traveling bags from foreign countries, and the disinfection and cleaning apparatus according to the present invention contributes to realizing a sanitary living environment within the country, which is also expected to become common among foreign lifestyles.

Thus, the summary of the present invention is as described in the following (1) to (10):
(1) a method of disinfection and cleaning, characterized in that the method includes a step of removing dust and grit and bacteria and the like adhered to an underside of an object to be disinfected and cleaned by contact with a disinfecting and cleaning fluid by means of a cleaning and removal means,
(2) the method of disinfection and cleaning according to (1), wherein the object to be disinfected and cleaned is a bag or the like with casters or the like or a conveyed object,
(3) the method of disinfection and cleaning according to (1), characterized in that the method further includes, prior to or subsequent to the step of removing dust and grit and bacteria and the like adhered to the underside of the object to be disinfected and cleaned, a space disinfection step in which a germicide is sprayed on the entire object to be disinfected and cleaned,
(4) the method of disinfection and cleaning according to (1) or (3), characterized in that the method further includes a step of removing the germicide or a different type of cleaning agent that remains adhered to the object to be disinfected and cleaned,
(5) the method of disinfection and cleaning according to (1), wherein the cleaning and removal means is a rotary brush or rotary artificial grasses,
(6) a disinfection and cleaning apparatus, characterized in that the apparatus includes a disinfection and cleaning apparatus main unit; a cleaning and removal means provided in the disinfection and cleaning apparatus main unit; a space on which an object to be disinfected and cleaned can be placed, the space being provided at a position where the object to be disinfected and cleaned can come into contact with the cleaning and removal means; and a sealing means causing the disinfection and cleaning apparatus main unit to be in a hermetically sealed state,
wherein the cleaning and removal means includes a pool unit for storing a disinfecting and cleaning fluid, formed in the bottom of the disinfection and cleaning apparatus main unit; a cleaning mechanism placed in the pool unit; and
a drive source for driving the cleaning mechanism to cause the cleaning mechanism to come into contact with the object to be disinfected and cleaned,
(7) the disinfection and cleaning apparatus according to (6), wherein the disinfection and cleaning apparatus main unit includes a space disinfection means inside for spraying a germicide,
(8) the disinfection and cleaning apparatus according to (6), characterized in that the cleaning mechanism includes: at least two driving rollers disposed in a longitudinal direction separated from each other; and a rotary belt structure or a rotary caterpillar structure extended by the driving rollers, which can come into contact with an underside of the object to be disinfected and cleaned,
(9) the disinfection and cleaning apparatus according to (6), characterized in that the rotary belt structure or the rotary caterpillar structure has a brush unit or an artificial grass structure mounted on a surface thereof,
(10) the disinfection and cleaning apparatus according to (6), wherein the space disinfection means includes: a spray nozzle for spraying a germicide into the interior space of the disinfection and cleaning apparatus main unit, the spray nozzle being formed by perforating an inside wall surface of the disinfection and cleaning apparatus main unit; and a germicide storage tank connected to the nozzle.

### Advantageous Effects of Invention

The present invention includes the configuration described above, and with respect to the first challenge, dust and grit and bacteria and the like adhered to the underside of the object to be disinfected and cleaned, especially casters of bags or the like, may be efficiently removed within a very short time by supplying the disinfecting and cleaning fluid to the underside of the object to be disinfected and cleaned by means of the cleaning and removal means without damaging the object to be disinfected and cleaned.

With respect to the second challenge, dust and grit, dirt, and bacteria and the like adhered to the underside of bags or the like, the bags or the like being the object to be disinfected and cleaned, may be removed within a short time, and bacteria and viruses adhered to the bags or the like may be removed by performing the space disinfection process on the entire bags or the like in addition to the underside of the bags or the like.

### DESCRIPTION OF EMBODIMENTS

Next, preferred embodiments of a method of disinfection and cleaning and a disinfection and cleaning apparatus according to the present invention will be described in detail. Note, however, that the present invention is not limited to the following preferred embodiments, and that any changes may be made hereto within the scope of the present invention.

### 1. Disinfection and Cleaning Method

The method of disinfection and cleaning according to the present invention includes removing, by washing, dust and grit, dirt, and bacteria and the like adhered to an underside of an object to be disinfected and cleaned by bringing a disinfecting and cleaning fluid into contact with the underside by means of a cleaning and removal means. In the method of disinfection and cleaning according to the present invention, the objects to be disinfected and cleaned include bags or the like such as traveling bags or suitcases although the objects to be disinfected and cleaned are not limited to those, and the method may be applicable to anything that needs disinfection and cleaning in daily life and that can withstand washing with the disinfecting and cleaning fluid.

The cleaning and removal means in the method of disinfection and cleaning according to the present invention is a member that can come into contact with the underside of the object to be disinfected and cleaned, which has an effect of scrapping dust and grit, dirt, and bacteria and the like off the underside of the object to be disinfected and cleaned and can deliver the disinfecting and cleaning fluid from a pool unit to supply the disinfecting and cleaning fluid to the underside of the object to be disinfected and cleaned. Specifically, the cleaning and removal means may be a rotary brush or rotary artificial grasses. The structures of the rotary brush and the rotary artificial grasses and the like will be described in description of a rotary brush structure and a rotary artificial grass structure to be described later.

In the method of disinfection and cleaning according to the present invention, the rotary brush and the rotary artificial grasses are served for removing fouling by being rotationally driven to travel in a lateral direction below the object to be disinfected and cleaned and to continuously perform a process of cleaning and removing dust and grit, dirt, and the like adhered to the underside of the object to be disinfected and cleaned. While conditions for the cleaning process, such as rotation rates of the rotary brush and the rotary artificial grasses, a temperature of the disinfecting and cleaning fluid, an amount of the disinfecting and cleaning fluid to be brought into contact with the underside, may vary depending on amounts of dust and grit, dirt, and the like adhered to the underside of the object to be disinfected and cleaned, the conditions may be adjusted such that the cleaning finishes within a predetermined period of time; the conditions may be adjusted such that a duration of the cleaning process is about five to twenty minutes.

According to the method of disinfection and cleaning according to the present invention, the rotary brush or the rotary artificial grasses are caused to come into contact with the underside of the object to be disinfected and cleaned at a tip portion thereof to release and supply, to the underside when the rotary brush or the rotary artificial grasses come into contact with the underside, the disinfecting and cleaning fluid in the pool unit placed below that has been absorbed by the rotary brush or the rotary artificial grasses while rotating. The supplied disinfecting and cleaning fluid soften the dust and grit, dirt, and the like stuck to the underside, and the rotary brush or the rotary artificial grasses scraps and removes the dust and grit, dirt, and the like off the object to be disinfected and cleaned.

By placing the object to be disinfected and cleaned on an installation space being provided above a rotary belt structure or a rotary caterpillar structure and driving the rotary belt structure or the rotary caterpillar structure to cause a face thereof located downward to absorb the disinfecting and cleaning fluid and to cause a face thereof located upward to come into contact with the underside of the object to be disinfected and cleaned, the disinfecting and cleaning fluid can be supplied and the dust and grit, dirt, and the like may be removed by a tip portion of a brush unit or an artificial grass structure of the rotary belt structure or the rotary caterpillar structure.

In addition, in the method of disinfection and cleaning according to the present invention, a disinfection process may be performed by, in conjunction with removing the dust and grit, dirt, and the like on the underside of the object to be disinfected and cleaned, spraying a germicide on the entire object to be disinfected and cleaned using a space disinfection means.

A space disinfection method may be performed by placing the object to be disinfected and cleaned in a closed room and spraying the germicide into a space inside the closed room. Although an amount of the germicide to be sprayed depends on the type of adhering bacteria, addition of a lethal dose of the germicide for bacteria for the entire space may work. In addition, while spraying of the germicide is enabled by releasing the germicide from an injection container storing the germicide, it is possible to use mists generated by directing gas through a germicide solution. The germicide is not particularly limited as long as properties thereof are suitable for space disinfection.

A more preferred embodiment of the method of disinfection and cleaning according to the present invention is defined as follows.

A method of disinfection and cleaning, characterized in that the method includes:
(1) a step of removing dust and grit and bacteria and the like adhered to an underside of an object to be disinfected and cleaned, by means of a rotary brush, with a disinfecting and cleaning fluid stored in a pool placed below the position where the object to be disinfected and cleaned is located; and
(2) a step of removing liquid such as a cleaning agent that remains on the underside of the object to be disinfected and cleaned, the underside having been cleaned in the aforementioned step.

The liquid such as a cleaning agent that remains is removed by performing a drying process, an evaporation process by means of spraying a gas, dry bath and the like. The disinfecting and cleaning fluid deteriorates as it is used, and impurities increase; accordingly, it is periodically replaced with a new disinfecting and cleaning fluid by means of a batch-type or continuous-type equipment.

In the method of disinfection and cleaning according to the present invention, more specifically, steps may include step-1 to step-3 as described below.

### Step-1

1. Removing grime on casters of a traveling bag
2. Cleaning and sanitizing an underside of the traveling bag with a disinfecting and cleaning fluid
3. Removing, by washing with water, grime inside the casters

### Step-2

Spraying a germicide on the entire traveling bag

### Step-3

Swabbing the entire traveling bag with a dry-bath towel

### 2. Disinfection and Cleaning Apparatus

The disinfection and cleaning apparatus according to the present invention includes a disinfection and cleaning apparatus main unit; a cleaning and removal means provided in the disinfection and cleaning apparatus main unit; a space on which an object to be disinfected and cleaned can be placed, the space being provided at a position where the object to be disinfected and cleaned can come into contact with the cleaning and removal means; and a front door causing the disinfection and cleaning apparatus main unit to be in a hermetically sealed state. The cleaning and removal means includes a pool unit formed in the bottom of the disinfection and cleaning apparatus main unit; and a cleaning mechanism placed in a disinfecting and cleaning fluid stored in the pool unit in an immersed state.

Each component of the disinfection and cleaning apparatus according to the present invention will be described below.

### <Cleaning and Removal Means>

The cleaning and removal means includes:
(1) the pool unit formed in the bottom of the disinfection and cleaning apparatus main unit;
(2) the cleaning mechanism placed in the pool unit; and
(3) a drive source for driving the cleaning mechanism.

The pool unit stores the disinfecting and cleaning fluid. The disinfecting and cleaning fluid is not particularly limited, and anything that can be obtained from the market may be selected for use.

In the pool unit, a drain valve is provided for replacing a deteriorated disinfecting and cleaning fluid with a new one. In addition, it is possible to determine the degree of deterioration of the disinfecting and cleaning fluid and to automatically perform replacement of the fluid accordingly.

The cleaning mechanism may be selected from the group consisting of a rotary belt structure and a rotary caterpillar structure, which will be described next.

### *Rotary Belt Structure

The rotary belt structure includes at least two driving rollers and a rotary belt extended by the two driving rollers.

As the rotary belt, a belt (hereinafter, referred to as “belt with brush bristles”) formed by directly planting brush materials on a back side of a belt body or a belt (hereinafter, referred to as “belt with a brush unit”) formed by attaching a sheet (hereinafter, referred to as “sheet planted with brush bristles”) constructed by directly planting brush materials on a sheet material on a back side of a belt body may be used. The sheet planted with brush bristles is detachably attached to the belt body. Attachment of the sheet planted with brush bristles to the belt body may be performed using a hook-and-loop fastener or other types of coupling means.

In other words, the rotary belt is formed by attaching the brush unit on a surface of the belt.

The rotary belt may be made of cloth or plastic, and the rotary belt is rotationally driven by the two driving rollers, and tips of the brush bristles are pressed to the underside of the traveling bag to remove dust and grit, dirt, and the like.

On a surface of the rotary belt that comes into contact with the driving rollers, a hook portion or a toothed portion may be formed, by which sliding on the driving rollers can be prevented.

### *Rotary Caterpillar Structure

The rotary caterpillar structure includes two driving rollers, a caterpillar rotationally driven by the driving rollers, and brush bristles or a brush unit, or an artificial grass structure mounted on a back side of the caterpillar.

The artificial grass structure is constructed by rafting a plurality of pile yarns on a base cloth in a suitable manner or filling spaces between a plurality of piles with some other resin material or the like, and tips of the piles remove dust and grit and the like. The filler is made of a water-absorbing material such as a water-absorbing resin, and can perform cleaning by releasing the disinfecting and cleaning fluid that the filler has absorbed in the pool unit in the disinfection and cleaning apparatus main unit.

The material of the pile yarns of the artificial grasses is preferably rigid for the purpose of the present invention, and the pile yarns may be selected from, e.g., polyolefin pile yarns or polyamide pile yarns.

### *Installation Space

The installation space in the disinfection and cleaning apparatus according to the present invention, as described in FIGS. 1 to 5 below and the like, is constructed from transverse bars and vertical bars, and the height of the installation space from the pool unit may be adjusted so that the tips of the brush bristles or the piles of the artificial grass structure of the rotary belt structure or the rotary caterpillar structure of the cleaning mechanism may be pressed to the underside of the object to be disinfected and cleaned. The height of the installation space is adjusted according to a type of the object to be disinfected and cleaned.

### <Space Disinfection Means>

The space disinfection means of the disinfection and cleaning apparatus according to the present invention includes:
an interior space of the disinfection and cleaning apparatus main unit; at least one spray nozzle formed in at least one hole, the hole being formed by perforating an inside wall surface for the interior space of the disinfection and cleaning apparatus main unit; and
a storage tank for supplying the disinfecting and cleaning fluid to the spray nozzle.

The space disinfection means of the disinfection and cleaning apparatus according to the present invention has the configuration as described above, and it is preferable to provide holes in which the spray nozzle is formed by perforation at a plurality of positions in the disinfection and cleaning apparatus interior space in order to keep the concentration of the germicide uniform.

### EXAMPLES

Embodiments of the present invention will be described on the basis of FIGS. 1 to 9.

FIG. 1 is a front view of a disinfection and cleaning apparatus 1 according to the present invention while FIG. 2 is a plan view, FIG. 3 is a right side view, and FIG. 4 is a left side view of the disinfection and cleaning apparatus 1. As illustrated in FIG. 1, the disinfection and cleaning apparatus 1 includes a disinfection and cleaning apparatus main unit (hereinafter, referred to as “apparatus main unit”) 10 and a disinfection and cleaning apparatus accessory unit (hereinafter, referred to as “accessory unit”) 20.

In the lower part of an interior space 101 of the apparatus main unit 10, a pool unit 102 in a form of a concave vessel is provided and a disinfecting and cleaning fluid B is stored in the pool unit 102. In the pool unit 102, a cleaning mechanism 103 is placed such that the lower half of the cleaning mechanism 103 is immersed in the disinfecting and cleaning fluid B by adjusting the amount of the disinfecting and cleaning fluid B to be poured.

The cleaning mechanism 103 includes driving rollers 1031 and 1031′, a supporting unit for the driving rollers 1033, driving roller shafts, a belt supporting member 1034, a rotary belt 1032, and a rotary belt 1032′.

As members constituting the installation space for placing the traveling bag A, transverse bars 1034 and vertical bars 1033 are illustrated.

The cleaning mechanism 103 is driven with the power from the variable motor 1043 transmitted to the driving rollers 1031′ via a rotating shaft 1042 and a power-transmitting gear 1041. The rotation rate is regulated by a speed controller 1044. With the rotation of the driving rollers, the rotary belt 1032 on which artificial grasses are mounted moves in the lateral direction, and piles of the artificial grasses come into contact with casters on an underside of a traveling bag A placed above the cleaning mechanism to remove fouling. A lower side of the rotary belt immersed in the disinfection and cleaning fluid B absorbs the disinfecting and cleaning fluid B, moves upward, and supplies the fluid B to the casters on the underside of the traveling bag to remove the dirt, dust, and general bacteria adhered to the back of the casters while performing cleaning.

The disinfecting and cleaning fluid B used in cleaning and removal may be renewed by opening a drain valve 30 to remove and replace the fluid B with a new cleaning fluid or by connecting an automatic changer to the drain valve 30 to automatically renew the fluid B. The disinfection and cleaning operation for the underside of the traveling bag A according to the present invention may be completed in about five to twenty minutes.

The disinfection and cleaning apparatus 1 according to the present invention includes the space disinfection means. The space disinfection means includes a storage tank 201 placed in the disinfection and cleaning apparatus accessory unit 20 and a spray nozzle, which is formed by perforating the inside wall surface of the apparatus main unit 10, for spraying a germicide C stored in the storage tank 201 into the inside of the apparatus main unit 10.

FIG. 5 is a perspective view of the disinfection and cleaning apparatus 1 according to the present invention with a front door of the apparatus main unit 10 opened. In FIG. 5, the installation space for the traveling bag A, which is constructed from transverse bars and vertical bars and provided above the cleaning mechanism 103, can be observed.

FIG. 6 is a perspective view of the disinfection and cleaning apparatus 1 according to the present invention with two traveling bags A placed on the installation space inside the apparatus main unit 10.

In FIG. 7, a nozzle 202 is formed by perforation in the disinfection and cleaning apparatus 1 according to the present invention.

FIG. 8 illustrates a state in which the rotary belt 1032 of the cleaning mechanism 103 is replaced with the caterpillar 1032′.

FIG. 9 illustrates a state in which an artificial grass structure 1032′-1 is mounted on an upper face of the caterpillar 1032-1 of the cleaning mechanism 103 of the disinfection and cleaning apparatus 1 according to the present invention.

### BRIEF DESCRIPTION OF DRAWINGS

FIG. 1 is a front view of a disinfection and cleaning apparatus according to one embodiment of the present invention;

FIG. 2 is a plan view of the disinfection and cleaning apparatus according to one embodiment of the present invention;

FIG. 3 is a right side view of the disinfection and cleaning apparatus according to one embodiment of the present invention;

FIG. 4 is a left side view of the disinfection and cleaning apparatus according to one embodiment of the present invention;

FIG. 5 is a front perspective view of the disinfection and cleaning apparatus according to one embodiment of the present invention with a front door opened;

FIG. 6 is a front view of a disinfection and cleaning apparatus according to one embodiment of the present invention, in which a traveling bag (an object to be disinfected and cleaned) is placed above a cleaning and removal means;

FIG. 7 is an explanatory diagram illustrating a pool unit of the disinfection and cleaning apparatus according to one embodiment of the present invention;

FIG. 8 is an explanatory diagram illustrating a caterpillar unit of a cleaning and removal means of the disinfection and cleaning apparatus according to one embodiment of the present invention; and

FIG. 9 is an explanatory diagram illustrating a state in which artificial grasses are attached to the caterpillar unit of the cleaning and removal means of the disinfection and cleaning apparatus according to one embodiment of the present invention.

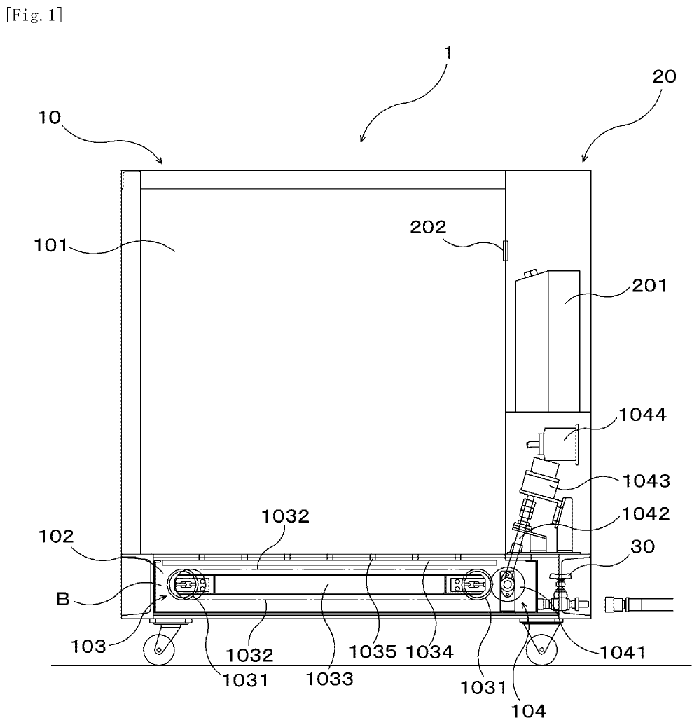

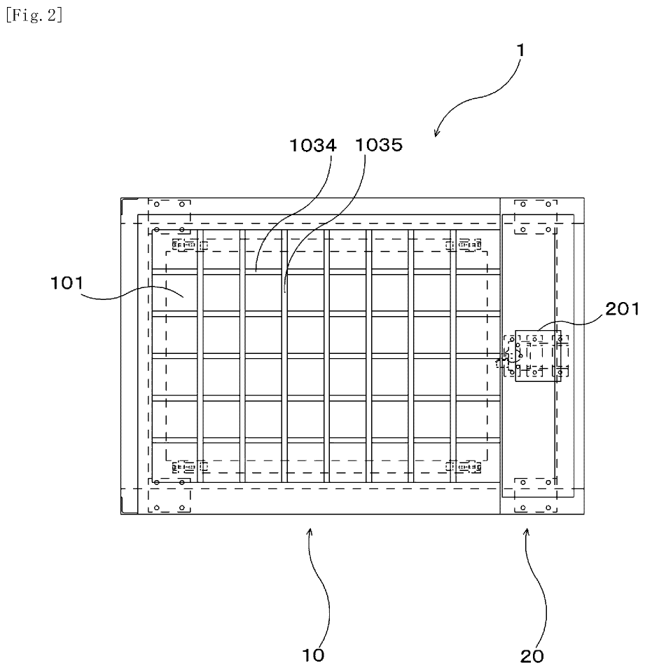

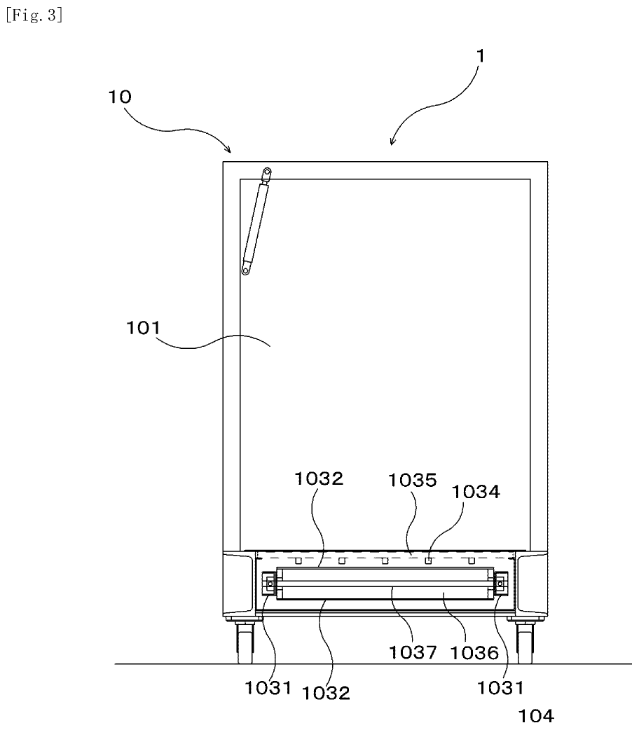

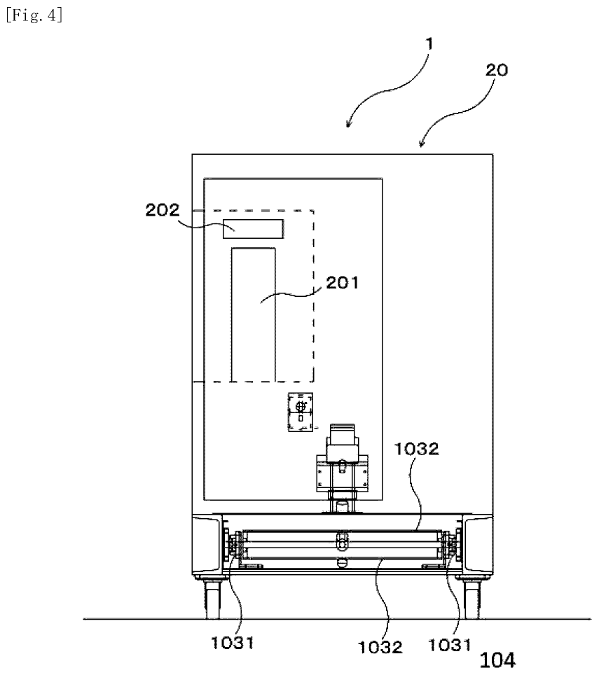

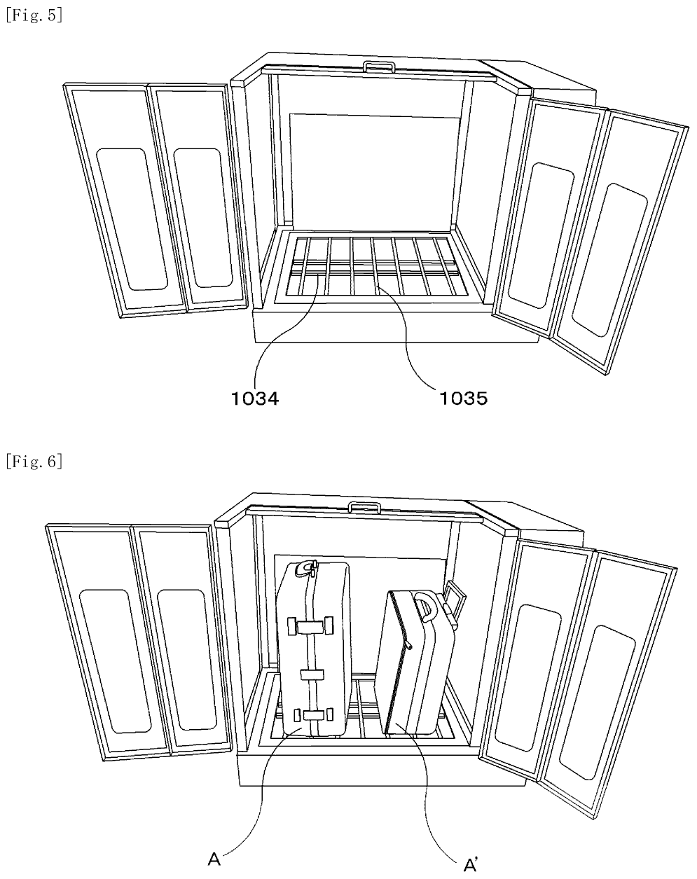

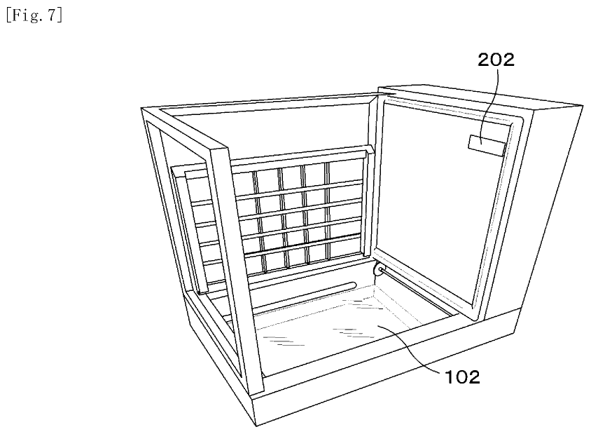

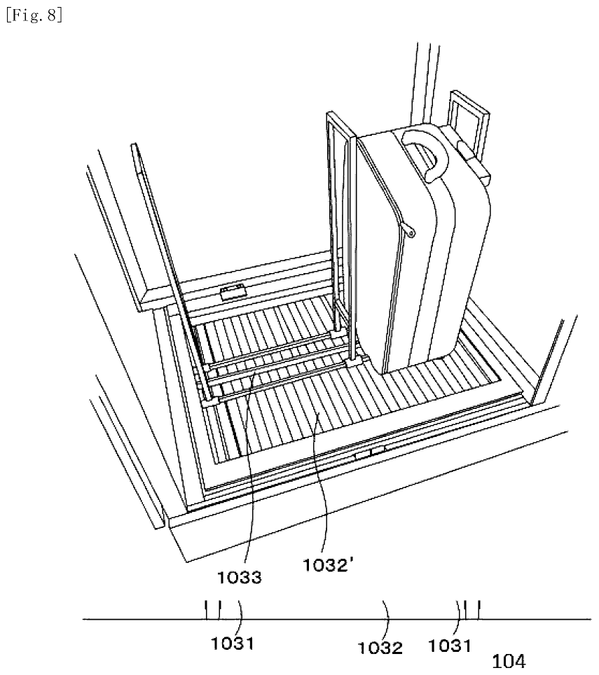

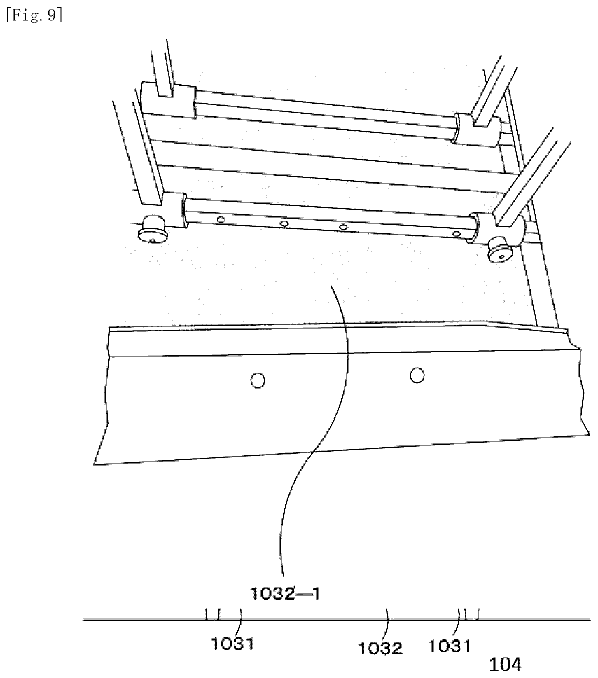

### REFERENCE SIGNS LIST

1 disinfection and cleaning apparatus
10 disinfection and cleaning apparatus main unit
101 interior space of the disinfection and cleaning apparatus main unit
102 pool unit of the disinfection and cleaning apparatus main unit
103 cleaning mechanism
1031 driving roller
1031′ driving roller
1032 one side of an upper portion of a rotary belt
1032′ one side of a lower portion of the rotary belt
1033 supporting unit for the driving rollers
1034 transverse bars of a traveling bag installation space
1035 vertical bars of the traveling bag installation space
104 power source for the driving rollers
1041 power-transmitting gear
1042 rotating shaft
1043 variable motor
1044 speed controller
20 disinfection and cleaning apparatus accessory unit
201 storage tank
202 spray nozzle

1. A method of disinfection and cleaning, comprising a step of removing dust and grit and bacteria adhered to an underside of an object to be disinfected and cleaned by contact with a disinfecting and cleaning fluid by means of cleaning and removal means.

2. The method of disinfection and cleaning according to claim 1, wherein the object to be disinfected and cleaned is a bag with casters or the like or a conveyed object.

3. The method of disinfection and cleaning according to claim 1, the method further comprising, prior to or subsequent to the step of removing dust and grit and bacteria adhered to the underside of the object to be disinfected and cleaned, a space disinfection step in which a germicide is sprayed on the entire object to be disinfected and cleaned.

4. The method of disinfection and cleaning according to claim 1, the method further comprising a step of removing the germicide or a different type of cleaning agent that remains adhered to the object to be disinfected and cleaned.

5. The method of disinfection and cleaning according to claim 1, wherein the cleaning and removal means is a rotary brush or rotary artificial grasses.

6. A disinfection and cleaning apparatus, comprising: a disinfection and cleaning apparatus main unit; cleaning and removal means provided in the disinfection and cleaning apparatus main unit; a space on which an object to be disinfected and cleaned can be placed, the space being provided at a position where the object to be disinfected and cleaned can come into contact with the cleaning and removal means; and sealing means causing the disinfection and cleaning apparatus main unit to be in a hermetically sealed state, the cleaning and removal means comprising a pool unit for storing a disinfecting and cleaning fluid, formed in the bottom of the disinfection and cleaning apparatus main unit;
a cleaning mechanism placed in the pool unit; and
a drive source for driving the cleaning mechanism in such a way that the cleaning mechanism causes the disinfecting and cleaning fluid to come into contact with the object to be disinfected and cleaned.

7. The disinfection and cleaning apparatus according to claim 6, wherein the disinfection and cleaning apparatus main unit further comprises space disinfection means.

8. The disinfection and cleaning apparatus according to claim 6, wherein the cleaning mechanism comprises: at least two driving rollers disposed in a longitudinal direction separated from each other; and a rotary belt structure or a rotary caterpillar structure extended by the driving rollers, which can come into contact with the object to be disinfected and cleaned to remove fouling.

9. The disinfection and cleaning apparatus according to claim 6, wherein the rotary belt structure and the rotary caterpillar structure have a brush unit or an artificial grass structure mounted on a surface thereof.

10. The disinfection and cleaning apparatus according to claim 7, wherein the space disinfection means includes: a spray nozzle for spraying a germicide into the inside of the disinfection and cleaning apparatus main unit, the spray nozzle being formed by perforating an inside wall surface of the disinfection and cleaning apparatus main unit; and a germicide storage tank connected to the nozzle by means of a tube.
